(19) United States
(12) Patent Application Publication
(10) Pub. No.: US 2025/0229085 A1
(43) Pub. Date: Jul. 17, 2025

(54) TRANSESOPHAGEAL VAGUS NERVE STIMULATION DEVICEPOSITIONING USING SENSED PHYSIOLOGICAL SIGNALS

(71) Applicant: Covidien LP, Mansfield, MA (US)

(72) Inventor: David J. Miller, Austin, TX (US)

(21) Appl. No.: 19/021,930

(22) Filed: Jan. 15, 2025

Related U.S. Application Data
(60) Provisional application No. 63/621,046, filed on Jan. 15, 2024.

(51) Int. Cl. A61N 1/36; A61N 1/05; A61N 1/372; G16H 20/40

(52) U.S. Cl. CPC A61N 1/36053; A61N 1/0507; A61N 1/36128; A61N 1/37247; G16H 20/40

(57) ABSTRACT

Example systems, devices, and techniques are disclosed for positioning one or more stimulation elements to deliver neurostimulation therapy transesophageally. An example system includes one or more electrodes disposed on a transesophageal stimulation device, the one or more electrodes including at least one sense electrode. The example systems includes one or more stimulation elements configured to deliver transesophageal stimulation to anatomy of the patient. The system includes sensing circuitry configured to generate a signal of a patient via the at least one sense electrode. The system includes processing circuitry communicatively coupled to the sensing circuitry. The processing circuitry is configured to obtain the signal from the sensing circuitry. The processing circuitry is configured to determine, based on the signal, a location of at least one stimulation element. The processing circuitry is configured to output an indication of the location.

### CROSS-REFERENCE TO RELATED APPLICATIONS

This application claims the benefit of and priority to U.S. Provisional Patent Application Ser. No. 63/621,046 filed Jan. 15, 2024, the entire disclosure of which is incorporated by reference herein.

### TECHNICAL FIELD

The disclosure relates to devices and techniques for positioning a stimulation device for stimulating a vagus nerve of a patient transesophageally.

### BACKGROUND

Neuromodulation by stimulation, including electrical stimulation, of the cervical, thoracic, and abdominal branches of the vagus nerve has been shown to be useful for a wide range of purposes.

### SUMMARY

In general, the disclosure is directed to devices, systems, and techniques for positioning a transesophageal stimulation device, such as one or more electrodes of a transesophageal stimulation system, for use in transesophageally stimulating a vagus nerve of a patient from within the esophagus of the patient. Because the vagus nerve is located in an anatomically challenging location to access, the vagus nerve may be challenging to stimulate without an invasive surgical procedure, which may be undesirable for situations including an acute illness, a short duration of stimulation, or when reduced time to stimulation is important to patient treatment. As such, it may be desirable to position a transesophageal stimulation device within the esophagus of a patient such that stimulation delivered by the transesophageal stimulation device may be delivered at an appropriate location for the delivery of efficacious stimulation. The system may be configured to determine a location for delivering stimulation from within the esophagus via a signal sensed from within the esophagus.

In one example, the disclosure is directed to a system including: one or more electrodes disposed on a transesophageal stimulation device, the one or more electrodes comprising at least one sense electrode; sensing circuitry configured to generate a signal of a patient via the at least one sense electrode; one or more stimulation elements configured to deliver transesophageal stimulation to anatomy of the patient; and processing circuitry communicatively coupled to the sensing circuitry, the processing circuitry being configured to: obtain the signal from the sensing circuitry; determine, based on the signal, a location within an esophagus of at least one stimulation element of the one or more stimulation elements; and output an indication of the location.

In another example, this disclosure is directed to a method including: obtaining, by processing circuitry and from sensing circuitry via at least one sense electrode of one or more electrodes disposed on a transesophageal stimulation device, a signal of a patient; determining, by the processing circuitry and based on the signal, a location of at least one stimulation element of one or more stimulation elements disposed on the transesophageal stimulation device, the at least one stimulation element being configured to deliver transesophageal stimulation to anatomy of the patient; and outputting, by the processing circuitry, an indication of the location.

In another example, this disclosure is directed to a system including: one or more electrodes disposed on a transesophageal stimulation device, the one or more electrodes comprising at least one sense electrode and at least one stimulation electrode configured to deliver a transesophageal stimulation signal to anatomy of the patient; sensing circuitry configured to generate a patient signal of a patient via the at least one sense electrode; stimulation circuitry configured to generate the transesophageal stimulation signal; one or more memories configured to store stimulation parameters that at least partially define the transesophageal stimulation signal; and processing circuitry communicatively coupled to the one or more memories, the sensing circuitry, and the stimulation circuitry, the processing circuitry being configured to: obtain the patient signal from the at least one sense electrode; determine, based on the patient signal, a location within an esophagus for delivering the transesophageal stimulation signal from the at least one stimulation electrode; and output an indication based on the location.

The details of one or more examples are set forth in the accompanying drawings and the description below. Other features, objects, and advantages of the disclosure will be apparent from the description and drawings, and from the claims.

The above summary is not intended to describe each illustrated example or every implementation of the present disclosure.

### BRIEF DESCRIPTION OF DRAWINGS

FIG. 1 is a conceptual diagram illustrating example anatomy of a patient.

FIG. 2 is a conceptual diagram illustrating an example transesophageal neurostimulation system according to one or more aspects of this disclosure.

FIGS. 3A-3C are block diagrams of example transesophageal neurostimulation systems according to one or more aspects of this disclosure.

FIG. 4 is a block diagram illustrating an example configuration of a computing device according to one or more aspects of this disclosure.

FIG. 5 is a flow diagram illustrating example positioning techniques according to one or more aspects of the present disclosure.

FIG. 6 is a flow diagram illustrating another example of positioning techniques according to one or more aspects of the present disclosure.

### DETAILED DESCRIPTION

The present disclosure is directed to devices, systems, and techniques for positioning a transesophageal neurostimulation device within the esophagus of a patient, for example, positioning one or more stimulation elements, such as stimulation electrodes, of the transesophageal neurostimulation device for transesophageal neurostimulation of a vagus nerve of a patient. The anatomical location of the vagus nerve makes the vagus nerve difficult to stimulate without an invasive surgical procedure. In an acute situation, such as during surgery or an abrupt illness, such as sepsis, stroke, myocardial infarction, traumatic brain injury, or when the required duration of stimulation is limited to short amount of time, it may be undesirable to undertake an invasive surgical procedure to implant a stimulation device. In such situations, the use of a transesophageal neurostimulation device may be appropriate.

In other situations, transesophageal neurostimulation of the vagus nerve can be used prophylactically perioperatively to prevent acute kidney injury (AKI) or postoperative ileus. Recent discoveries relating to vagus nerve stimulation have uncovered the nervous system involvement and control of the inflammatory response of the body. The nervous system senses inflammation, pathogens, and tissue damage, as well as modulates the response. One pathway of the nervous system is referred to as the cholinergic anti-inflammatory pathway (CAP). Animal and humans studies have shown the stimulating certain nerves, usually branches of the vagus nerve, can dampen the inflammatory response and associated cytokines. Stimulation has been investigated in the cervical vagus, the abdominal vagus, the auricular branch of the vagus in the ear, the sacral nerve, the tibial nerve, and others. Recent studies have shown that by varying the stimulation, inflammatory cytokines can be modulated up or down.

Implantable cervical vagus stimulators are commercially available for the treatment of epilepsy, but involve complex and invasive surgery to implant the stimulating electrode on the nerve. Other technologies attempt to stimulate the vagus transcutaneously with an external device, but those have shown limited success due to the distance from the skin surface to the vagus nerve.

A device or system configured to stimulate the vagus nerve without an invasive surgical procedure may be used to trigger the CAP. CAP has been shown to reduce excessive inflammation and would be useful for treating a variety of illnesses including, but not limited to: surgical or non-surgical acute kidney injury; postoperative ileus; postoperative cognitive decline or postoperative delirium; asthma; sepsis; bleeding control; myocardial infarction reduction; and dysmotility and obesity. Treating any of these diseases may improve patient outcomes by shortening length of hospital stays and reducing medical costs.

Many conditions can be caused by damage to tissue from an overreaction of the inflammatory process. One such condition is ischemia-reperfusion injury (IRI). In IRI, tissue experiences ischemia due to reduced or stopped blood supply, followed by reperfusion due to medical intervention or the body's healing response. When tissue experiences an IRI, the immune system reacts to the damaged or dead cells with an intense inflammatory response causing infarcted tissue and loss of long-term function. Reducing the inflammatory response during the ischemia or reperfusion can reduce the resulting infarct volume and improve function. Examples of common conditions that may lead to IRI include myocardial infarction (the abrupt blockage of coronary arteries leading to zones of infarcted heart tissue), acute ischemic stroke and transient ischemic attack (the blockage of blood vessels in the brain or leading to the brain), or AKI (abrupt loss of renal function due to an injury). AKI typically occurs in surgical patients and septic patients. AKI is distinct from chronic kidney disease, which is the gradual loss of kidney function. AKI can be caused by many things, but a common cause is reduced renal blood flow and/or renal blood oxygen extraction.

There are other common acute medical problems that involve an inappropriate over-reaction by the immune system. Some examples of such conditions include severe asthma attacks with may involve excessive mucus secretion and airway narrowing, sepsis (some forms of sepsis may be driven by the immune system over-reacting, which may be referred to as a “cytokine storm”), or post-operative ileus. For example, after abdominal surgeries, it is common for patients to have ileus, or the inability of the intestine (bowel) to contract normally and move waste out of the body. Ileus may be caused by an inflammatory response in the bowel due to surgical manipulation.

Other conditions manifest as an imbalance in the sympathetic/parasympathetic balance. If the imbalance is decreased parasympathetic tone, the imbalance can cause temporary cardiac arrhythmias.

All of the above conditions can be treated (e.g., reduced symptoms or improved clinical outcomes) by stimulating the vagus nerve. Vagus nerve stimulation may reduce inflammatory damage from IRI, return inflammation to a normal level and prevent the hyperinflammatory response, and/or restore a healthy, normal parasympathetic/sympathetic balance.

The inflammatory response of a patient to an acute health problem may be a major risk factor to complications during the acute health problem, such as surgery or an acute illness. It may not be desirable to engage in an invasive surgical procedure to implant a stimulation device during the time the patient is experiencing the acute health problem, as that may be a further risk factor to complications during the acute health problem. Therefore, there may be a need for devices, systems, and techniques for stimulating the cervical, thoracic, or abdominal vagus branches that is relatively easy and quick to use, such as a transesophageal stimulation system. Such devices or techniques may be used for short-term stimulation, such as during an acute health problem, such as surgery or during an abrupt illnesses, such as sepsis. In order to effectively administer transesophageal stimulation, a transesophageal stimulation device may need to be located such that one or more stimulation elements are located at an appropriate stimulation location within the esophagus. As such, it may be desirable to determine whether one or more stimulation elements are located at such location(s). This disclosure describes examples of devices, systems, and techniques for determining a location of one or more stimulation elements of a transesophageal stimulation device.

The techniques described include acutely stimulating the vagus transesophageally. In one example, the stimulation device is placed in the esophagus lumen and stimulates the vagus through the wall of the esophagus. In order to deliver efficacious stimulation to the vagus nerve transesophageally, the stimulation electrodes, and/or other stimulation element(s), should be properly positioned along the esophagus in an acceptable location. The lower esophagus may curve in multiple planes, and/or include anatomic variations across patients. Such curves and/or variations may make it more difficult to properly position the stimulation electrodes using conventional techniques, such as inserting an elongated element of a transesophageal neurostimulation system to a certain depth of insertion in the esophagus of a patient or using imaging techniques during insertion of the transesophageal neurostimulation system into a patient. For example, anatomic measurements are currently used clinically to guide the insertion depths of nasogastric tubes. One example of this anatomic measurement guide is the nose-earlobe-xiphoid distance (NEX) calculation. However, even when attempting to insert the distal tip of the nasogastric tube in the stomach of a patient using a NEX calculation, the distal tip of the nasogastric tube does not end up in the stomach of the patient in about 10% of the cases. Transesophageal vagus nerve stimulation requires more accurate placement than that offered by the use of imaging techniques and/or NEX calculations to be optimally effective.

For the purpose of this disclosure, “above” refers a direction towards the mouth of a patient relative to the esophagus of the patient, and “below” refers to a direction towards the stomach of the patient relative to the esophagus of the patient.

A location which may be a desirable location to deliver transesophageal stimulation may be in the lower esophagus, above the stomach, and below where the esophagus is close (e.g., closest) to the heart. While this potential stimulation zone is typically several centimeters long, it may be difficult to determine the location of this potential stimulation zone precisely based on anatomy measurements or x-ray or other imaging techniques. Another possible stimulation location would be at, or near, a location where the esophagus passes through the diaphragm. In this location, the vagus is primarily organized into an anterior and posterior branch that are both attached to the outer layer of the esophagus. The esophagus is thin at only about 1 mm thick, even thinner if distended. Stimulation may be relatively easily performed at this distance.

According to the techniques of this disclosure, a system or a clinician may use information from a signal, such as the shape or morphology of an ECG signal and/or a diaphragmic EMG signal, to determine an appropriate location for the stimulation electrodes and/or other stimulation elements of the transesophageal stimulation device for the delivery of transesophageal stimulation. The signal may be sensed by one or more electrodes disposed within the esophagus.

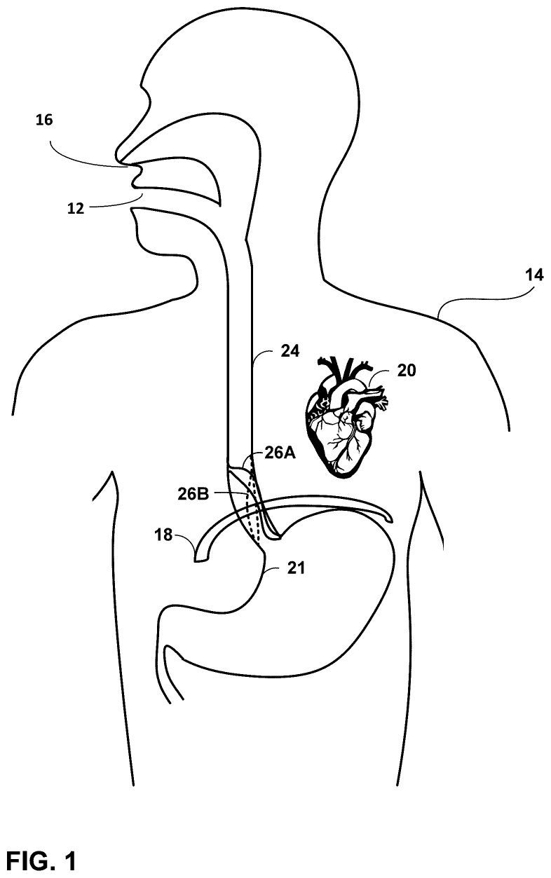

FIG. 1 is a conceptual diagram illustrating an example location of a vagus nerve. Patient 14 is depicted having stomach 21, esophagus 24, heart 20, and diaphragm 18. Mouth 12 and nasal cavity 16 are connected to esophagus 24 and may provide access to esophagus 24 for a transesophageal neurostimulation system (not shown). Also depicted are representations of branches of the vagus nerve, namely anterior branch 26A of the vagus nerve or posterior branch 26B of the vagus nerve. A transesophageal neurostimulation device may deliver neurostimulation to one or more of anterior branch 26A of the vagus nerve or posterior branch 26B of the vagus nerve via one or more electrodes and/or other stimulation elements of the transesophageal neurostimulation device disposed within esophagus 24 of patient 14. Sense electrodes of the transesophageal neurostimulation device may be used to sense physiological signals, such as an ECG or an EMG which may be used to position the transesophageal neurostimulation device. In some examples, when a position of the transesophageal neurostimulation device is not optimal or desirable, a system, which may include the transesophageal neurostimulation device may output visual and/or auditory indicator(s) for a clinician to reposition the transesophageal neurostimulation device to improve or optimize the position of the transesophageal neurostimulation device for stimulation. In some examples, the indicator(s) may form a feedback mechanism for the clinician positioning the transesophageal neurostimulation device. For example, sense electrodes may sense an ECG of heart 20 or an EMG within esophagus 24 in an area where esophagus 24 passes through diaphragm 18. Such sensed signals may be used to position stimulation electrodes and/or other stimulation elements at a location with esophagus 24 to provide efficacious stimulation therapy to the vagus nerve.

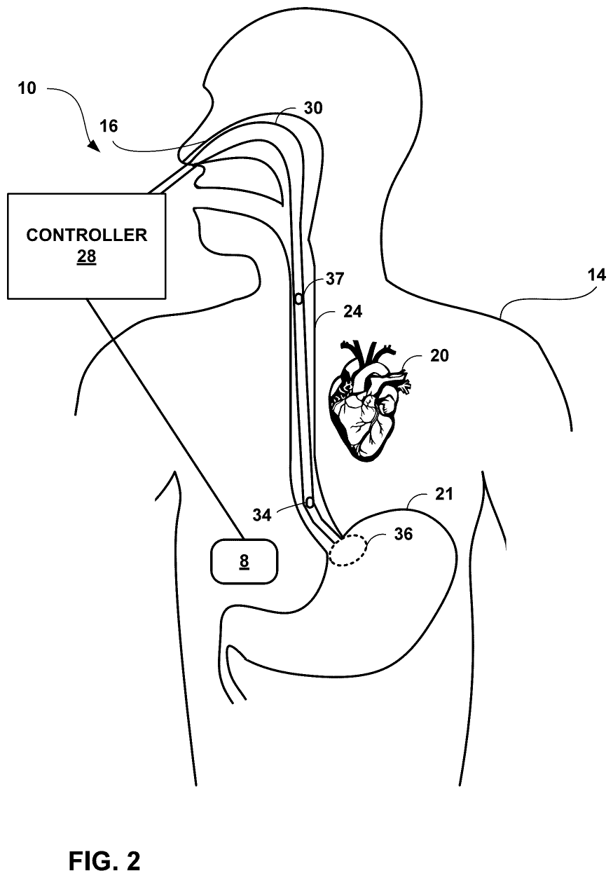

FIG. 2 is a conceptual diagram illustrating an example transesophageal neurostimulation system 10 according to the techniques of this disclosure. A distal end of transesophageal neurostimulation system 10 may be introduced into esophagus 24 through either nasal cavity 16 (as shown) or mouth 12 and stimulate the vagus nerve (not shown in FIG. 2) through the wall of esophagus 24. One possible location of the delivery of stimulus would be at or near where 24 esophagus passes through diaphragm 18 (not shown in FIG. 2), or caudal from diaphragm 18 of patient 14. At this location, the vagus nerve is primarily organized into anterior branch 26A and posterior branch 26B (FIG. 1) that are both attached to the outer layer of esophagus 24. Esophagus 24 may be thin, around only about 1-3 mm thick, and even thinner if esophagus 24 is distended. Thus, transesophageal neurostimulation system 10 may deliver stimulation (e.g., electrical, chemical, pharmaceutical, mechanical, and/or thermal) through the wall of esophagus 24 and to a portion of the vagus nerve. In some examples, transesophageal neurostimulation system 10 may be used to stimulate one or more of branches of the vagus nerve, roots of the vagus nerve, ganglia of the vagus nerve, or plexus of the vagus nerve. In some examples, transesophageal neurostimulation system 10 may be biased, such as being bent or weighted, in such a manner as to position electrodes of transesophageal neurostimulation system 10 at locations more likely to be near the vagus nerve, such as anterior branch 26A and/or posterior branch 26B. In some examples, electrodes may stand elevated relative to a surface of elongated member 30 and/or expandable member 36. In some examples, transesophageal neurostimulation system 10 may include a steerable or deflectable device configured to indent, appose, or penetrate electrodes of transesophageal neurostimulation system 10 into an inner wall of esophagus 24. For example, the steerable or deflectable device may be flexible for insertion into patient 14, but include a bias to a shaft of the device (such as elongated member 30) and/or a direction of deflection that facilitates the positioning of the stimulation electrodes and/or other stimulation elements at locations more likely to be near the vagus nerve.

Esophagus 24 is located between the spinal column and heart 20. The anterior of esophagus 24 is adjacent to heart 20. In some examples, to reduce or avoid inadvertent heart stimulation, transesophageal neurostimulation system 10 may be configured to direct stimulation towards posterior branch 26B or posterior trunk of the vagus nerve. Sensing electrodes of electrode(s) 34 may be used to sense an ECG of patient 14 and controller 28 may determine the posterior direction based on the sensed ECG signal. While shown as a single electrode for purposes of simplicity, electrode 34 may include more than one electrode. For example, ECG signals sensed from electrodes facing the posterior of patient 14 may sense a lower amplitude ECG then electrodes facing the anterior of patient 14. In some examples, elongated member 30 may be shaped in such a manner as to automatically orient the stimulation electrode(s) posteriorly.

Transesophageal neurostimulation system 10 includes controller 28, elongated member 30, and optionally expandable member 36. Controller 28 may be configured to control neurostimulation being delivered to the vagus nerve (not shown in FIG. 2) of patient 14. For example, controller 28 may include processing circuitry, telemetry circuitry, and memory. The telemetry circuitry may be configured for wireless or wired communication. In some examples, controller 28 may also include stimulation circuitry configured to generate a stimulation signal. In other examples, the stimulation circuitry may be located in a portion of transesophageal neurostimulation system 10 that is internal to patient 14.

Controller 28 may be an example of a computing device. In some examples, external controller 28 may include a clinician programmer or patient programmer. In some examples, controller 28 may be a device for inputting stimulation programs or stimulation parameters into transesophageal neurostimulation system 10. In some examples, controller 28 may be a wearable communication device, with a therapy request input integrated into a key fob or a wristwatch, handheld computing device, smart phone, computer workstation, or networked computing device. Controller 28 may include a user interface that is configured to receive input from a user (e.g., patient 14, a caretaker, or a clinician). In some examples, the user interface includes, for example, a keypad and a display, which may for example, be a liquid crystal display (LCD) or light emitting diode (LED) display. In some examples, the user interface may include a turnable knob or a representation of a turnable knob. The keypad may take the form of an alphanumeric keypad or a reduced set of keys associated with particular functions. Controller 28 may additionally or alternatively include a peripheral pointing device, such as a mouse, via which a user may interact with the user interface. In some examples, a display of controller 28 may include a touch screen display, and a user may interact with controller 28 via the display.

A user, such as a clinician, a patient, or a caregiver, may also interact with controller 28 to communicate with transesophageal neurostimulation system 10. Such a user may interact with controller 28 to retrieve physiological or diagnostic information from one or more of a sensor, processing circuitry, or memory that may be located on or in a portion of transesophageal neurostimulation system 10 that is intended to be within patient 14 during stimulation. The user may also interact with controller 28 to program transesophageal neurostimulation system 10, e.g., select values for the stimulation parameter with which transesophageal neurostimulation system 10 generates and delivers stimulation and/or the other operational parameters of transesophageal neurostimulation system 10, such as one or more stimulation parameters (e.g., pulse amplitude, pulse width, pulse frequency, pulse burst duration, electrode combination, etc.), user requested periods for stimulation or periods to prevent stimulation, or any other such user customization of therapy.

For example, the user may use controller 28 to receive and/or retrieve information from transesophageal neurostimulation system 10 relating to a signal sensed from within the patient, such as an ECG of patient 14 and/or an EMG of patient 14 to better position elongated member 30 such that stimulation electrodes of electrodes 34 may be appropriately positioned to deliver stimulation to the vagus nerve. The user may also use controller 28 to retrieve information from sensors of elongated member 30, such as a heartrate, a heart rate variability over time, respiration rate, vagus nerve sensed activity, temperature, or the like. As another example, the user may use controller 28 to retrieve information from transesophageal neurostimulation system 10 relating to the performance or integrity of transesophageal neurostimulation system 10. In some examples, this information may be presented to the user as an alert if a system condition that may affect the efficacy of therapy is detected. A system condition may include an output of an error code of the transesophageal neurostimulation system 10, such as for exceeding a temperature, for example.

In some examples, the user may use controller 28 to retrieve (or controller 28 may send) information regarding the development of undesirable conditions with respect to ethe use of transesophageal neurostimulation system 10. Examples of undesirable conditions may include a heartrate, a heart rate variability over time, respiration rate, vagus nerve sensed activity, temperature, or the like, meeting or exceeding a respective threshold, relative differences between any combination of such sensed information meeting or exceeding a threshold, a rate of change of such sensed information meeting or exceeding a threshold, and/or the like.

As mentioned above, the system may use an ECG to locate or navigate elongated member 30 within the esophagus to a target location for positioning one or more stimulation elements. Generally, at least two sense electrodes are required to collect an ECG signal. The electrical potential measured between these sense electrodes may have a different shape or morphology based on the position of the sense electrodes relative to heart 20. As such, controller 28 may use the sensed ECG signals to determine a location of the sense electrodes within esophagus 24 of patient 14. Controller 28 may determine the location of stimulation electrodes, based on the location of the sense electrodes. For example, when the distance between one or more of the sense electrodes and the stimulation electrodes is known, controller 28 may determine the location of the stimulation electrodes based on the location of the one or more of the sense electrodes.

In some examples, all of the sense electrodes may be located or positioned on elongated member 30. In other examples, at least one sense electrode may be located or positioned on elongated member 30 and at least one sense electrode may be located or positioned on return pad 8 which may be secured to the skin of patient 14. Transesophageal neurostimulation system 10 may be designed so that stimulation electrodes (or other stimulation elements) may be appropriately positioned within esophagus 24 of patient 14 when the sense electrodes are used to sense the ECG signal. For example, a distance and/or orientation between the sense electrode(s) and stimulation electrodes may be known, such when the location of the sense electrode(s) within esophagus 24 is determined based on the ECG signal, the location of the stimulation electrodes may be determined.

In some examples, one or more of the same electrodes used for stimulating (e.g., stimulation electrodes) may also be used to determine the location within esophagus 24. With this locational information, feedback to the user may indicate whether the transesophageal neurostimulator should be advanced more caudal or retracted cranial, for example. In some examples, one or more of the stimulation electrodes may be the same as one or more of the sense electrodes. In such examples, controller 28 may locate a defined spot with one or more of the sense/stimulation electrodes of electrodes 34 and a clinician may then move the one or more sense/stimulation electrodes of electrodes 34 a defined distance, e.g., by moving elongated member 30. For example, controller 28 may use sense electrode(s) to determine a location in esophagus 24 that is closest to the left atrium of heart 20. Controller 28 (or a clinician) may determine an insertion depth for patient 14. For example, controller 28 may determine that elongated member 30 is inserted a certain number of centimeters into patient 14 when the electrode(s) are closest to the left atrium of heart 20 based on the ECG signal. The clinician may then insert elongated member 30 an additional distance. This additional distance may be predetermined and/or could range from 1 cm to 15 cm to move the stimulating electrodes of electrodes 34 from an area in esophagus 24 near heart 20 to an area near the vagus nerve.

As mentioned above, the ECG sensed from one or more electrodes on the shaft of the stimulation device may have a different shape or morphology based on their position relative to the heart in the esophagus. For example, the p-wave may change shape or even invert as an electrode is positioned on different sides of heart 20 (e.g., above heart 20 or below heart 20). The sensed ECG signal may have a certain morphology, such as a largest peak-to-peak amplitude, a largest volume under the curve, a particular shape, a particular frequency, or the like, when sensed at the location within esophagus 24 near the left atrium of heart 20 than at other locations within esophagus 24. Controller 28 may compare sensed ECG signals from various locations within esophagus 24 to each other, to one or more stored morphology templates, to one or more thresholds, and/or the like to determine when the sense electrodes are at the location within esophagus 24 near the left atrium of heart 20.

In some examples, one or more stimulation elements may be appropriately positioned in esophagus 24 using a sensed EMG signal. For example, such a sensed EMG signal may be an EMG signal sensed from the diaphragm during a respiratory cycle of patient 14 or another muscle near the esophagus. A location where esophagus 24 passes through diaphragm 18 may be an appropriate location for transesophageal vagus nerve stimulation. In some examples, such as when stimulation elements are the same as sensing electrodes, when at this location, controller 28 may output a visual and/or auditory indicator to inform a clinician that the one or more stimulation elements are appropriately positioned in esophagus 24. For example, one or more sense electrodes of electrodes 34 may be used to sense an EMG signal within esophagus 24 caused by movement of diaphragm 18 to affect breathing of patient 14. Sensing circuitry or processing circuitry may identify signal characteristics of diaphragm 18 from the EMG signal using one or more filters and/or algorithms to differentiate from other electrical signals within the patient. Controller 28 may, based on the sensed EMG signal, determine a location within esophagus 24 where esophagus 24 passes through diaphragm 18. For example, the sensed EMG signal may have a certain morphology, such as a largest peak-to-peak amplitude, a largest volume under the curve, a particular shape, a particular frequency, or the like, when sensed at the location within esophagus 24 where esophagus 24 passes through diaphragm 18 than at other locations within esophagus 24. This morphology may be indicative of the location to diaphragm 18. Controller 28 may compare sensed EMG signals from various locations within esophagus 24 to each other, to one or more stored morphology templates, to one or more thresholds, and/or the like to determine when the sense electrodes are at the location within esophagus 24 where esophagus 24 passes through diaphragm 18. In some examples, controller 28 may determine which sense electrode(s) and/or which stimulation element(s) are closest to the location within esophagus 24 where esophagus 24 passes through diaphragm 18.

In an example where the sense electrodes are also used as stimulation electrodes, when controller 28 determines the location of the sense electrodes to be at the location within esophagus 24 where esophagus 24 passes through diaphragm 18, the stimulation electrodes may be at an appropriate location within esophagus 24 for stimulating the vagus nerve. As such, controller 28 may output an indication that electrodes 34 are appropriately located for stimulation and/or may automatically control stimulation circuitry to begin delivering stimulation via stimulation electrodes of electrodes 34. In an example where the sense electrodes are different than the stimulation electrodes, controller 28 may determine an insertion depth corresponding to the location where esophagus 24 passes through diaphragm 18 and/or may determine an additional distance which a clinician should move elongated member 30 to position the stimulation electrodes at the location within esophagus 24 where esophagus 24 passes through diaphragm 18. Once the clinician moves elongated member 30 the additional distance, the stimulation electrodes of electrodes 34 may be at an appropriate location for delivering vagus nerve stimulation.

By using an ECG and/or diaphragmatic EMG signal to locate stimulation element(s) of a transesophageal neurostimulation system, the techniques of this disclosure may allow for quicker placement and a simpler neurostimulation device than one using expandable member 36 in stomach 21 of patient 14 to attempt to locate the stimulation element(s) in an appropriate location for delivery of vagus nerve stimulation. For example, in the treatment of stroke, the faster the stimulation begins (and therefore, the faster the stimulation element(s) of the transesophageal neurostimulation system are appropriately located), the better the patient outcome will likely be. Controller 28 may provide visual and/or auditory indicator(s) to guide a clinician in positioning stimulation element(s) in the appropriate location for delivery of the vagus nerve stimulation and if the position of the stimulation element(s) changes during the delivery of transesophageal neurostimulation, controller 28 may provide visual and/or auditory indicator(s) to guide the clinician to reposition the stimulation element(s).

Controller 28 may display indications of the location of the sense electrode(s), the stimulation element(s), and/or the appropriateness of the location of elongated member 30 in esophagus 24 of patient 14 for use by a clinician in initiating stimulation, relocating elongated member 30 (and/or sense electrodes/stimulation electrodes), and/or terminating stimulation.

Patient 14 or a clinician may, for example, use a keypad or touch screen of controller 28 to request transesophageal neurostimulation system 10 to deliver or terminate the stimulation. For example, patient 14 may use controller 28 to provide a therapy request to control the delivery of electrical stimulation “on demand,” e.g., when patient 14 deems the second stimulation therapy desirable. This request may be a therapy trigger event used to terminate electrical stimulation.

Controller 28 may provide a notification to patient 14 or a clinician when the electrical stimulation is being delivered or notify patient 14 of the prospective termination of the electrical stimulation. In such examples, controller 28 may display a visible message, emit an audible alert signal or provide a somatosensory alert (e.g., by causing a housing of controller 28 to vibrate). In other examples, the notification may indicate when therapy is available (e.g., a countdown in minutes, or indication that therapy is ready). In some examples, controller 28 may provide a notification to patient 14 or a clinician if the position of the stimulation element(s) changes during the delivery of transesophageal neurostimulation and provide visual and/or auditory indicator(s) to guide the clinician to reposition the stimulation element(s) to an appropriate position.

In the example where controller 28 includes stimulation circuitry, elongated member 30 may include conductors configured to conduct the stimulation signal from the stimulation circuitry of controller 28 to stimulation electrode(s) of electrode(s) 34. Elongated member 30 may define a lumen configured to permit the removal or introduction of substances from patient 14. For example, the lumen may permit the introduction of food, drink, medication, or the like from external of the patient into esophagus 24 or stomach 21. The lumen may also permit the aspiration of the contents of stomach 21. In this way, the presence of a lumen may eliminate the need for placement of a traditional feeding tube. In such cases, the portion of elongated member 30 elongate tube external to patient 14 may allow for user interface for such purposes similar to traditional nasogastric tubes.

While transesophageal neurostimulation system 10 is primarily discussed as being configured to deliver electrical stimulation, in some examples, transesophageal neurostimulation system 10 may be configured to deliver other types of stimulation to the vagus nerve via one or more stimulation elements. For example, transesophageal neurostimulation system 10 may be configured to deliver mechanical stimulation at relatively low frequency, such as through vibration, or at relatively higher frequencies, such as ultrasound. In such a case, rather than, or in addition to, having stimulation electrodes, elongated member 30 may include one or more stimulation elements including a vibration causing device, such as a haptic device, and/or an ultrasound device, which may be configured to deliver mechanical stimulation to the vagus nerve. In some examples, transesophageal neurostimulation system 10 may be configured to deliver thermal stimulation to the vagus nerve. In such a case, rather than, or in addition to, having stimulation electrodes, elongated member 30 include one or more stimulation elements including a thermal device configured to deliver heating or cooling toward the vagus nerve to stimulate the vagus nerve. In some examples, transesophageal neurostimulation system 10 may be configured to deliver chemical or pharmaceutical stimulation to the vagus nerve. In such a case, rather than, or in addition to, having stimulation electrodes, elongated member 30 may include one or more stimulation elements which may be configured to release a bolus of one or more chemicals or pharmaceuticals under the control of controller 28.

When present, optional expandable member 36 may be configured to expand in stomach 21 of patient 14, under the control of a clinician, in such a manner as to anchor elongated member 30 to stomach 21 such that elongated member 30 does not move in a direction away from stomach 21. For example, once a clinician has positioned elongated member 30 such that stimulation electrodes of electrode(s) 34 are in a desired position for stimulation, the clinician may deploy expandable member 36.

In some examples, expandable member 36 may include a balloon or other expandable structure, such as a mechanically expandable structure that includes struts and/or linkages that enables expansion (e.g., similar to a stent or cage). In some examples, expandable member 36 may include one or more of a spiral, a helix, a partial spiral, a plurality of spirals, a plurality of helixes, or a plurality of partial spirals. In some examples, electrodes may stand elevated relative to a surface of expandable member 36.

Elongated member 30 and expandable member 36 (when present) may be sized in the range of from 5 to 25 French or 8 to 18 French when expandable member 36 is in a non-expanded or collapsed state to enable relatively easy introduction of elongated member 30 and expandable member 36 within nasal cavity 16 or mouth 12 of patient 14. Relatively easy may mean as easy as a standard nasogastric tube and may require similar nursing skills to place trans-nasally as a standard nasogastric tube. In some examples, in order to reduce sliding friction between system 10 and patient 14 during insertion of a portion of system 10 into patient 14, elongated member 30 and/or expandable member 36 may be lubricated. In some examples, the lubrication may be contained within packaging that encloses at least a portion of system 10, may be pre-lubricated, or, in some examples, system 10 may be configured to self-lubricate. For example, controller 28 may include a lubrication pump that pump lubricant onto an exterior surface of elongated member 30 and/or expandable member 36. In some examples, elongated member 30 and/or expandable member 36 may define a lubricating lumen which may carry lubricant from the lubrication pump to an exterior surface of elongated member 30 and/or expandable member 36 via lubrication openings. In other examples, a lubrication lumen of elongated member 30 and/or expandable member 36 may be prefilled with lubricant and the pressure exerted upon elongated member 30 and/or expandable member 36 by esophagus 24 during insertion of elongated member 30 into esophagus 24 may cause the prefilled lubricant to be discharged via the lubrication openings to the exterior surface of elongated member 30. In other examples, a coating may be applied to elongated member 30 and/or expandable member 36 which may become lubricious when in contact with saliva or mucus of patient 14.

In some examples, in addition to, or alternatively, in order to reduce patient discomfort caused by system 10, elongated member 30 and/or expandable member 36 may be pre-coated with a local anesthetic such as lidocaine. In some examples, the local anesthetic may be included in the packaging that encloses at least a portion of system 10. In some examples, the local anesthetic may be combined with a lubricant.

Transesophageal neurostimulation system 10 may deliver electrical stimulation to patient 14 by generating and delivering a programmable electrical stimulation signal (e.g., in the form of electrical pulses or an electrical waveform) to a target a therapy site near electrodes 34 disposed, in some examples, on an outer surface of elongated member 30. The distal end of transesophageal neurostimulation system 10 may be inserted into patient 14 in such a manner as to locate electrodes 34 near the vagus nerve of patient 14. A clinician may use sensed physiological signals, such as ECGs and/or EMGs, to position stimulation electrodes of electrodes 34 to be in an appropriate location to deliver efficacious stimulation. For example, controller 28 may provide visual and/or auditory indicator(s) to guide a clinician in positioning stimulation element(s) in the appropriate location for delivery of the vagus nerve stimulation. Elongated member 30 and expandable member 36 (when present) may be constructed of biocompatible materials.

Electrodes 34 may be configured to be circumferentially and or longitudinally separated from each other on an outer surface of elongated member 30. In some examples, transesophageal neurostimulation system 10 may be configured to deliver a stimulation signal to the vagus nerve of patient 14 via electrodes 34 in a cycled manner. For example, the delivery of the stimulation signal may move over time between different electrode combinations of electrodes 34. In this manner, a clinician may not need to circumferentially align any particular electrodes of electrodes 34 with branches of the vagus nerve. In some examples, electrodes 34 may operate in a bipolar or multi-polar configuration. For example, one or more electrodes of electrodes 34 may be configured as anodes and one or more of electrodes 34 may be configured as cathodes. Such a configuration is different than a unipolar configuration which would include an electrode located at a position relatively remote from the vagus nerve. In other examples, electrodes 34 may operate in unipolar configuration. In such a case, the return electrode(s) may be located on a portion of elongated member 30 in esophagus 24, distant from electrode(s) 34, such as return electrode 37, or a return pad 8 on the skin of the patient. In some examples, return pad 8 on the skin may be placed on the abdomen near the lower esophagus to steer the current on a path the goes through the vagus nerve.

In some examples, transesophageal neurostimulation system 10 may include between 2 and 16 electrodes, inclusively. In some examples, the electrodes may be used for delivering different stimulation therapies or other electrical stimulations to respective stimulation sites within patient 14 or for monitoring at least one physiological marker of patient 14. For example, a set of electrodes may deliver stimulation at a first frequency to a first branch of the vagus nerve while a different set of electrodes may deliver stimulation at a second frequency to a second branch of the vagus nerve. In another example, a set of electrodes may deliver stimulation at a first frequency to a first location of first branch of the vagus nerve while a different set of electrodes may deliver stimulation at a second frequency to a second location of the first branch of the vagus nerve. This may allow for directional stimulation, such as blocking in a distal direction and stimulating in a proximal direction for an afferent stimulation. In some examples, the first frequency may be on the order of 1 Hz to 200 Hz for delivery of therapy (e.g., about 20 Hz) and the second frequency may be on the order of 1 kHz to 50 kHz for creating a nerve block (e.g., between about 10 kHz to about 20 kHz). In some examples, there may be separate electrodes of electrodes 34 for delivering blocking and stimulating, and these separate electrodes may be arranged axially along a transesophageal neurostimulation device rather than, or in addition to, circumferentially around the device.

Transesophageal neurostimulation system 10 is an example of a transesophageal neurostimulation system that may use the techniques of this disclosure or to which the techniques of this disclosure may be applied. These techniques are applicable to a wide variety of transesophageal neurostimulation systems. Examples of transesophageal neurostimulation systems and devices that may be used with the techniques of this disclosure includes those set forth in U.S. Patent Publication 2023/0026849, entitled Transesophageal Vagus Nerve Stimulation, filed on Jul. 13, 2022; U.S. Provisional Patent Application No. 63/489,356, entitled Multi-Site Neuromodulation, filed on Mar. 9, 2023; and U.S. Provisional Patent Application No. 63/489,352, entitled Transesophageal Neurostimulation, filed on Mar. 9, 2023, the entire content of each of which is incorporated herein by reference.

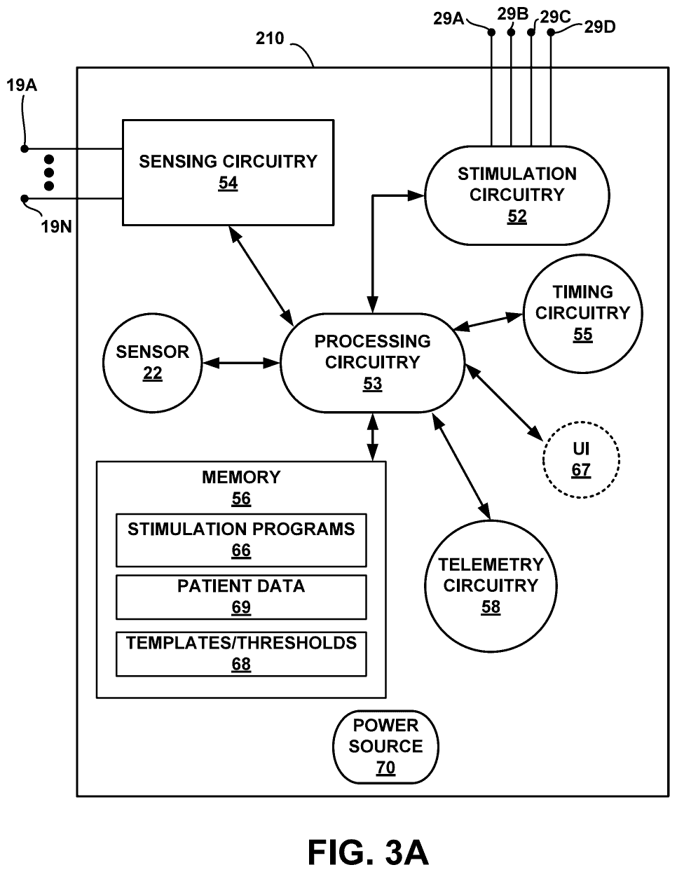

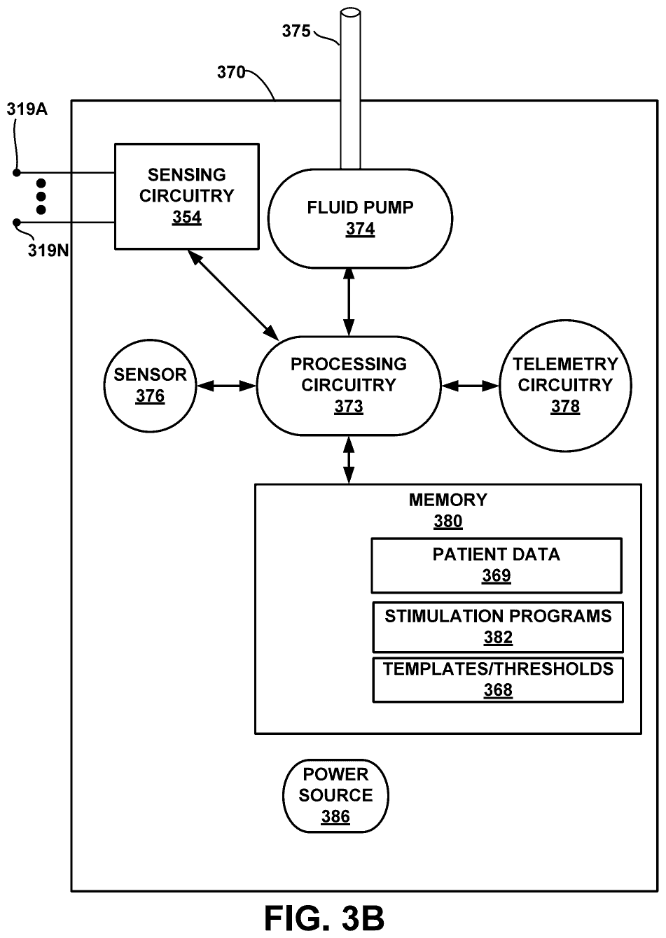

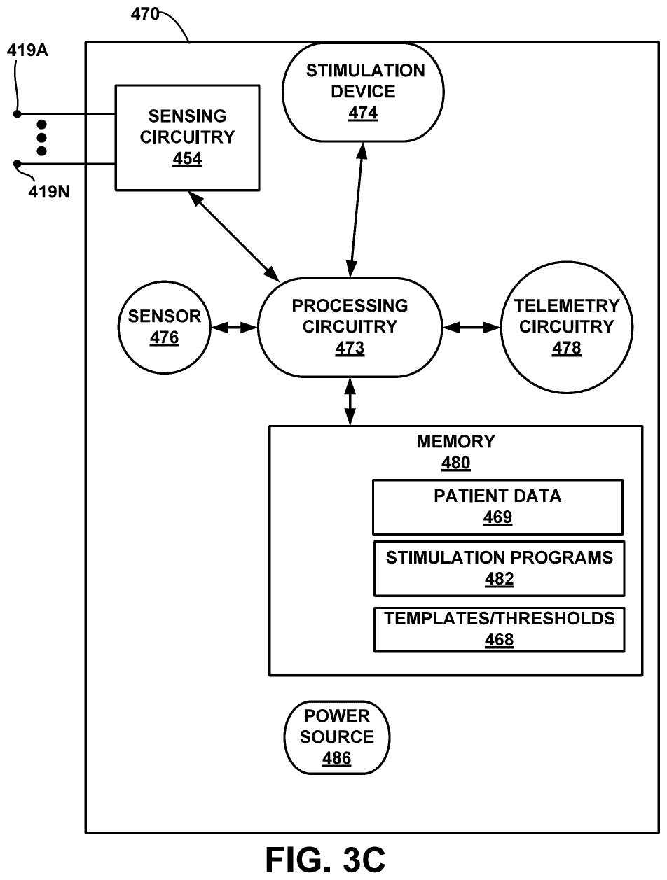

FIGS. 3A, 3B, and 3C are block diagrams of example transesophageal neurostimulation systems according to one or more aspects of this disclosure. In some examples, each of the transesophageal neurostimulation systems of FIGS. 3A-3C may be one device. In other examples, each of the transesophageal neurostimulation systems of FIGS. 3A-3C may represent more than one device or a collection of devices, such as transesophageal neurostimulation system 10 (FIG. 2).

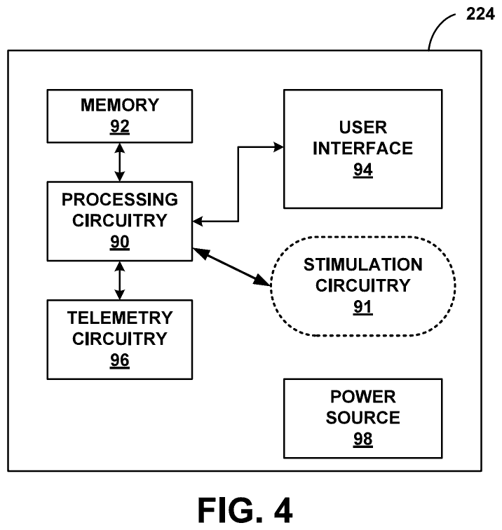

In the example of FIG. 3A, transesophageal neurostimulation system 210 may include stimulation circuitry 52 configured to generate a stimulation signal, processing circuitry 53, telemetry circuitry 58, timing circuitry 55, memory 56, sensor 22 (which may be configured to sense one or more physiological parameters of patient 14), and sensing circuitry 54 (which may be configured to sense an ECG signal and/or an EMG signal of patient 14). Transesophageal neurostimulation system 210 may also include one or more electrodes, such as electrodes 29A-29D (collectively “electrodes 29”), and electrodes 19A-19N (collectively “electrodes 19”). Electrodes 29 and 19 may be examples of electrodes 34. For example, sensing circuitry 54 may sense an ECG signal and/or an EMG signal as described herein via any of electrodes 29 and/or 19. In some examples, the electrodes sensing the ECG signal and/or EMG signal may be the same electrodes or different electrodes than those providing the stimulation. In some examples, transesophageal neurostimulation system 210 may also include a user interface (UI 67) which may function similarly to user interface 94 described in more detail in the discussion of FIG. 4 below. In examples where the sense electrodes may also be stimulation electrodes, stimulation circuitry 52 and sensing circuitry 54 may be combined or may be coupled to electrodes 19 and/or 29 via switching circuitry (not shown).

Sensor 22 may comprise a patient motion sensor that generates a signal indicative of patient posture state, orientation, or activity level. In some examples, transesophageal neurostimulation system 210 may use sensor 22 (which may include an accelerometer) to identify posture states of patient 14. Processing circuitry 53 may use the posture state, the ECG signal, and/or the EMG signal to determine a position of one or more electrodes 29 and may use the position to determine which electrode combination or other stimulation parameters to use for stimulation. For example, stimulation programs 66 may include predetermined programs for supine, prone, lateral, or other common surgical positions. By knowing the surgical position, the likely position of the vagus branches on patient 14, and the orientation of the electrodes of transesophageal neurostimulation system 210, processing circuitry 53 may automatically select the electrodes to be used for stimulation. Transesophageal neurostimulation system 210 may also operate in a closed-loop manner by controlling stimulation parameters and the delivery of stimulation is response to sensed physiologic parameters such as heart rate, heart rate variability, respiration rate, vagus nerve sensed activity, temperature, EMG, activity level of patient 14, or other measures. These physiological parameters may be sensed by sensor 22 and/or sensing circuitry 54. For example, processing circuitry 53 may control stimulation circuitry 52 to titrate and optimize the neurostimulation therapy based on the sensed physiological parameters.

In some examples, the neurostimulation could be delivered to the vagus nerve (in neck, chest, or abdomen). While the target tissue for the delivery of stimulation is primarily discussed herein as being the vagus nerve, other potential locations of interest may include the sacral nerve, the pudendal nerve, the splenic nerve, the splanchnic nerve, tibial nerve, or other peripheral nerves.

In some examples, the physiological parameters may be sensed by external devices, such as pulse oximetry sensors, Near Infrared Spectroscopy (NIRS), Bispectral Index processed electroencephalogram (EEG), external EMG electrodes, EEG electrodes, wearable activity tracker, cameras, depth-sensing cameras, or other sensors. In some examples, physiological parameters may be measured by anesthesia equipment such as a multi-parameter monitor (MPM) or respirator. In some examples, the physiological parameters may be sensed by an implantable sensor such as in a pacemaker or cardiac monitor. By using sensed physiological parameters to control the stimulation, processing circuitry 53 may maximize, optimize, or otherwise improve the stimulation of the cholinergic anti-inflammatory pathway (CAP). CAP has been shown to reduce excessive inflammation and would be useful for treating a variety of illness including, but not limited to: surgical or non-surgical acute kidney injury, postoperative ileus, postoperative cognitive decline or Postoperative delirium; asthma; sepsis; bleeding control; myocardial infarction reduction; dysmotility and obesity. Treating any of these diseases may improve patient outcomes by shortening hospital length of stays and reducing costs.

According to some examples, processing circuitry 53 identifies changes to the patient's physiological state that are relevant to desired changes in neurostimulation. For example, processing circuitry 53 may control stimulation circuitry 52 to generate a stimulation signal that is gated to the respiratory cycle or heartbeat. Vagus nerve stimulation may be more effective when gated to certain physiological activities. For example, it may enhance the potency of the vagus nerve stimulation if the stimulation is gated to be during a phase of respiration, such as the exhalation phase of respiration. For example, the respiration cycle of patient 14 may be accurately detected with pulse oximetry signal analysis, the diaphragmic EMG signal, or an accelerometer in the device. In some examples, processing circuitry 53 may use other physiologic activities to gate the stimulation. For example, processing circuitry 53 may determine heart rate or circadian rhythms and gate the stimulation signal based on the heart rate, phase of a cardiac cycle, or a phase of a circadian rhythm.

Monitoring other physiological parameters may also serve to enhance safety. For example, stimulating the cervical vagus may depress the heart rate of patient 14. Processing circuitry 53 may be configured to control stimulation circuitry 52 to stop stimulation or lower a stimulation intensity if the heart rate declined below a threshold. Similarly, processing circuitry 53 may monitor sensed vital signs to monitor pain in an unconscious person. Processing circuitry 53 may be configured to control stimulation circuitry 52 to stop stimulation or lower a stimulation intensity if processing circuitry 53 determines that increasing pain is not associated with surgery or changes in anesthesia. In some examples, processing circuitry 53 may use one or more of the sensed parameters to balance between a parasympathatic and sympathetic tone in patient 14.

In some examples, transesophageal neurostimulation system 210 may be configured to stimulate muscles or motor neurons and to determine an upper limit of stimulation amplitude through one or more feedback techniques, such as a sensed EMG. Other potential feedback techniques which transesophageal neurostimulation system 210 may employ include sensed resulting evoked compound action potential (ECAP) for each stimulation, an accelerometer signal indicative of whether muscular movement is occurring, or patient reported sensations. For example, transesophageal neurostimulation system 210 may include a device, such as a hand-held device, which patient 14 may provide, through a user interface, an indication of a sensory threshold or discomfort threshold for various electrode combinations.

In general, transesophageal neurostimulation system 210 may comprise any suitable arrangement of hardware, alone or in combination with software and/or firmware, to perform the techniques attributed to transesophageal neurostimulation system 210 and processing circuitry 53, stimulation circuitry 52, sensing circuitry 54, and telemetry circuitry 58 of transesophageal neurostimulation system 210. In various examples, transesophageal neurostimulation system 210 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. Transesophageal neurostimulation system 210 also, in various examples, may include a memory 56, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although processing circuitry 53, stimulation circuitry 52, sensing circuitry 54, and telemetry circuitry 58 are described as separate circuitry, in some examples, processing circuitry 53, stimulation circuitry 52, sensing circuitry 54, and telemetry circuitry 58 are functionally integrated. In some examples, processing circuitry 53, stimulation circuitry 52, sensing circuitry 54, and telemetry circuitry 58 correspond to individual hardware units, such as microprocessors, ASICs, DSPs, FPGAs, or other hardware units. In further examples, any of processing circuitry 53, stimulation circuitry 52, sensing circuitry 54, and telemetry circuitry 58 may correspond to multiple individual hardware units such as microprocessors, ASICs, DSPs, FPGAs, or other hardware units.

Memory 56 stores stimulation programs 66 that specify stimulation parameter values for the electrical stimulation provided by transesophageal neurostimulation system 210. Stimulation programs 66 may also store information relating to determining and using physiological parameters, such as threshold values. In some examples, transesophageal neurostimulation system 210 may deliver stimulation therapy based on one or more physiological markers. In other examples, transesophageal neurostimulation system 210 may deliver stimulation therapy that is not based on one or more physiological markers. In some examples, memory 56 also stores patient data 69 which may include sensed physiological parameters. Patient data 69 may also include timing information which may be associated with the sensed physiological parameters. In some examples, memory 56 also stores templates/thresholds 68 which may include one or more ECG and/or EMG templates, one or more ECG and/or EMG thresholds, or the like, which may be used by processing circuitry 53 to determine a location of sensing electrode(s) and/or stimulation element(s) (such as stimulation electrodes) as described herein.

Generally, stimulation circuitry 52 generates and delivers electrical stimulation under the control of processing circuitry 53. In some examples, processing circuitry 53 controls stimulation circuitry 52 by accessing memory 56 to selectively access and load at least one of stimulation programs 66 to stimulation circuitry 52. For example, in operation, processing circuitry 53 may access memory 56 to load one of stimulation programs 66 to stimulation circuitry 52. In other examples, stimulation circuitry 52 may access memory 56 and load one of the stimulation programs 66. In some examples, the electrical stimulation signal generated and delivered by stimulation circuitry 52 may be above around 10 Hz to avoid activating muscular contraction.

In some examples, stimulation programs 66 may include stimulation programs that are configured to facilitate different effects. For example, stimulation circuitry may use different stimulation programs to generate different electrical stimulation signals to cause different effects. In some examples, stimulation circuitry 52 may generate an electrical stimulation signal in the range of about 1 to 200 Hz to reduce inflammation in patient 14 (e.g., around 20 Hz) or generate an electrical stimulation signal in the range of about 1 kHz to about 50 kHz to block and increase an inflammatory response (e.g., between about 10 kHz to about 20 kHz).

By way of example, processing circuitry 53 may access memory 56 to load one of stimulation programs 66 to stimulation circuitry 52 for delivering the electrical stimulation to patient 14. A clinician or patient 14 may select a particular one of stimulation programs 66 from a list using a programming device, such as controller 28 (FIG. 2) or computing device 224 (FIG. 4). Processing circuitry 53 may receive the selection via telemetry circuitry 58. Stimulation circuitry 52 delivers the electrical stimulation to patient 14 according to the selected program for an extended period of time, such as minutes, hours, days, or until patient 14 or a clinician manually stops or changes the program.

Stimulation circuitry 52 delivers electrical stimulation according to stimulation parameters. In some examples, stimulation circuitry 52 delivers electrical stimulation in the form of electrical pulses. In such examples, relevant stimulation parameters may include a voltage amplitude, a current amplitude, a pulse rate, a pulse width, a duty cycle, a duty cycle of the stimulation ON/OFF periods, or the combination of electrodes 29 that stimulation circuitry 52 uses to deliver the stimulation signal. In other examples, stimulation circuitry 52 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes 29 stimulation circuitry 52 uses to deliver the stimulation signal.

In some examples, processing circuitry 53 may control stimulation circuitry 52 to deliver or terminate the electrical stimulation based on patient or clinician input received via telemetry circuitry 58. Telemetry circuitry 58 includes any suitable hardware, firmware, software or any combination thereof for communicating with another device, such as computing device 224 (FIG. 4) or another device external to transesophageal neurostimulation system 210. Under the control of processing circuitry 53, telemetry circuitry 58 may receive communications, e.g., patient or clinician input, from and send communications, e.g., an alert, to an external device, such as the computing device of FIG. 4, discussed hereinafter. In the example, where transesophageal neurostimulation system 210 is representative of transesophageal neurostimulation system 10, the external device may use an antenna (not shown) when communicating, which may be internal and/or external. Processing circuitry 53 may provide the data to be sent to the external device and the control signals for the telemetry circuit within telemetry circuitry 58, and receive data from telemetry circuitry 58. For example, processing circuitry 53 may control telemetry circuitry 58 to output one or more indications described herein, such as indications that a stimulation element(s), such as stimulation electrodes 29 (and/or 19 when also acting as stimulation electrodes) are located appropriately for vagus nerve stimulation or that elongated member 30 should be moved to locate the stimulation element(s) in a proper position for stimulating the vagus nerve.

Generally, processing circuitry 53 may control telemetry circuitry 58 to exchange information with the external device or another device external to transesophageal neurostimulation system 210 wirelessly or wired. Processing circuitry 53 may transmit operational information and patient data 69 and receive stimulation programs or stimulation parameter adjustments via telemetry circuitry 58. Also, in some examples, transesophageal neurostimulation system 210 may communicate with other devices, such as stimulators, control devices, or sensors, via telemetry circuitry 58.

In some examples, power source 70 delivers operating power to the components of transesophageal neurostimulation system 210. In some examples, power source 70 may include a battery and a power generation circuit to produce the operating power. In some examples, the battery may be rechargeable to allow extended operation. Recharging may be accomplished through proximal inductive interaction between an external charger and an inductive charging coil within transesophageal neurostimulation system 210. In other examples, an external inductive power supply may transcutaneously power transesophageal neurostimulation system 210 whenever electrical stimulation is to occur. In some examples, power source 70 may be coupled to an external power source, such as an outlet on a hospital wall.

A stimulation program of stimulation programs 66 may define various parameters of the stimulation waveform and electrode configuration which result in a predetermined stimulation intensity being delivered to the targeted vagus nerve. In some examples, the stimulation program defines parameters for at least one of a current or voltage amplitude of the stimulation signal, a frequency or pulse rate of the stimulation, the shape of the stimulation waveform, a duty cycle of the stimulation, a pulse width of the stimulation, a duty cycle of the stimulation ON/OFF periods, and/or the combination of electrodes 34 and respective polarities of the subset of electrodes 34 used to deliver the stimulation. Together, these stimulation parameter values may be used to define the stimulation intensity (also referred to herein as a stimulation intensity level). In some examples, if stimulation pulses are delivered in bursts, a burst duty cycle also may contribute to stimulation intensity. Also, independent of intensity, a particular pulse width and/or pulse rate may be selected from a range suitable for causing the desired therapeutic effect after stimulation is terminated and, optionally, during stimulation. In addition, as described herein, a period during which stimulation is delivered may include on and off periods (e.g., a duty cycle or bursts of pulses) where even the short inter-pulse durations of time when pulses are not delivered are still considered part of the delivery of stimulation. A period during which transesophageal neurostimulation system 210 withholds stimulation delivery is a period in which no stimulation program is active for transesophageal neurostimulation system 210 is not tracking pulse durations or inter-pulse durations that occur as part of the electrical stimulation delivery scheme). In addition to the above stimulation parameters, the stimulation may be defined by other characteristics, such as a time for which stimulation is delivered, a time for which stimulation is terminated, and times during which stimulation is withheld.

As shown in FIG. 3B, transesophageal neurostimulation system 370 is similar to transesophageal neurostimulation system 210 of FIG. 3A, but transesophageal neurostimulation system 370 delivers neurostimulation to patient 14 in the form of chemicals and/or pharmaceuticals instead of electrical stimulation. Transesophageal neurostimulation system 370 includes processing circuitry 373 (e.g., similar to processing circuitry 53 of FIG. 3A), fluid pump 374 coupled to catheter 375 (which may be an example of a stimulation element), sensing circuitry 354 (e.g., similar to sensing circuitry 54), electrodes 319A-319N (e.g., similar to electrodes 19), sensor 376 (e.g., a pressure sensor, accelerometer, or other sensor (which may be similar to sensor 22 of FIG. 3A), telemetry circuitry 378 (e.g., similar to telemetry circuitry 58 of FIG. 3A), memory 380 (e.g., similar to memory 56 of FIG. 3A), and power source 386 (e.g., similar to power source 70 of FIG. 3A).

Fluid pump 374 may include a chemical and/or pharmaceutical reservoir and a chemical and/or pharmaceutical pump that moves the chemical and/or pharmaceutical from the reservoir, through catheter 375, and out to patient 14. In some examples, fluid pump 374 may move a chemical and/or pharmaceutical from a reservoir external to transesophageal neurostimulation system 370. In some examples, transesophageal neurostimulation system 370 may include both a chemical and/or pharmaceutical reservoir, pump, and electrical stimulation generator. Memory 380 may include stimulation programs 382, patient data 369, and templates/thresholds 368 (e.g., similar to templates/thresholds 68). Stimulation programs 382 may include instructions for chemical and/or pharmaceutical delivery. In some examples, such instructions may cause the delivery of stimulation in a closed-loop manner such that delivery of stimulation is based on physiological parameters of patient 14. Patient data 369 may include sensed physiological parameters of the patient or the like. Processing circuitry 373 may control fluid pump 374 to deliver a bolus of one or more chemicals and/or one or more pharmaceuticals to patient 14 based on a stimulation program of stimulation programs 366.

As shown in FIG. 3C, transesophageal neurostimulation system 470 is similar to transesophageal neurostimulation system 210 of FIG. 3A, but transesophageal neurostimulation system 470 delivers neurostimulation to patient 14 thermally, or mechanically, e.g., in the form of low frequency mechanical stimulation (e.g., vibration) and/or in the form of high frequency mechanical stimulation (e.g., ultrasound). Transesophageal neurostimulation system 470 includes processing circuitry 473 (e.g., similar to processing circuitry 53 of FIG. 3A), stimulation device 474 (which may be an example of a stimulation element), sensing circuitry 454 (e.g., similar to sensing circuitry 54), electrodes 419A-419N (e.g., similar to electrodes 19), sensor 476 (e.g., a pressure sensor, accelerometer, or other sensor (which may be similar to sensor 22 of FIG. 3A), telemetry circuitry 478 (e.g., similar to telemetry circuitry 58 of FIG. 3A), memory 480 (e.g., similar to memory 56 of FIG. 3A), and power source 486 (e.g., similar to power source 70 of FIG. 3A).

Stimulation device 474 may include a device configured to deliver thermal or mechanical stimulation to patient 14 at a relatively low frequency, such as a haptic device, and/or a device configured to deliver mechanical stimulation to patient 14 at a relatively high frequency, such as an ultrasound emitter. In some examples, stimulation device 474 may deliver mechanical stimulation via a transducer (not shown) extending from the housing of transesophageal neurostimulation system 470 or from a surface of the housing of transesophageal neurostimulation system 470. The positioning techniques of this disclosure may be used to position stimulation device or the transducer in an appropriate location to deliver efficacious transesophageal stimulation.

In some examples, transesophageal neurostimulation system 470 may include a thermal stimulation device, a low frequency mechanical stimulation device and/or high frequency mechanical stimulation device, as well as a pharmaceutical reservoir, pump, and/or an electrical stimulation generator. Memory 480 may include stimulation programs 482, patient data 469, and templates/thresholds 468 (e.g., similar to templates/thresholds 68). Stimulation programs 482 may include instructions for delivery of mechanical and/or thermal stimulation. In some examples, such instructions may cause the delivery of stimulation in a closed-loop manner such that delivery of stimulation is based on physiological parameters of patient 14. Patient data 469 may include sensed physiological parameters of the patient or the like. Processing circuitry 473 may control stimulation device 474 to deliver mechanical and/or thermal stimulation to patient 14 based on a stimulation program of stimulation programs 366.

FIG. 4 is a block diagram illustrating an example configuration of a computing device. Computing device 224 may be an example of controller 28. Computing device 224 may include notebook computer, a smart phone, a workstation, a key fob, or a wearable device, for example. As illustrated in FIG. 4, computing device 224 may include a processing circuitry 90, memory 92, user interface 94, telemetry circuitry 96, and power source 98. Memory 92 may store program instructions that, when executed by processing circuitry 90, cause processing circuitry 90 and computing device 224 to provide the functionality ascribed to controller 28 throughout this disclosure. In some examples, computing device 224 may include stimulation circuitry 91 which may function similarly to stimulation circuitry 52 of FIG. 3A. In general, computing device 224 comprises any suitable arrangement of hardware, alone or in combination with software and/or firmware, to perform the techniques attributed to computing device 224, and processing circuitry 90, user interface 94, and telemetry circuitry 96 of computing device 224. In various examples, computing device 224 may include one or more processors, such as one or more microprocessors, DSPs, ASICs, FPGAs, or any other equivalent integrated or discrete logic circuitry, as well as any combinations of such components. Computing device 224 also, in various examples, may include a memory 92, such as RAM, ROM, PROM, EPROM, EEPROM, flash memory, a hard disk, a CD-ROM, comprising executable instructions for causing the one or more processors to perform the actions attributed to them. Moreover, although processing circuitry 90 and telemetry circuitry 96 are described as separate circuitry, in some examples, processing circuitry 90 and telemetry circuitry 96 are functionally integrated. In some examples, processing circuitry 90 and telemetry circuitry 96 and telemetry circuitry 58 correspond to individual hardware units, such as microprocessors, ASICs, DSPs, FPGAs, or other hardware units. In other examples, any of processing circuitry 90 and telemetry circuitry 96 and telemetry circuitry 58 may correspond to multiple individual hardware units, such as microprocessors, ASICs, DSPs, FPGAs, or other hardware units.

Memory 92 may store program instructions that, when executed by processing circuitry 90, cause processing circuitry 90 and computing device 224 to provide the functionality ascribed to computing device 224 throughout this disclosure. In some examples, memory 92 may further include program information, e.g., stimulation programs defining the neurostimulation, similar to those stored in memory 56 of transesophageal neurostimulation system 210. The stimulation programs stored in memory 92 may be downloaded into memory 56 of transesophageal neurostimulation system 210. Memory 92 may store templates/thresholds, such as templates/thresholds 68 of FIG. 3A.

In certain examples, computing device 224 includes a user interface 94 that allows the patient to provide input. Patient 14 may, additionally or alternatively, request a change in stimulation program or settings through user interface 94.

User interface 94 may include a button or keypad, lights, a speaker for voice commands, a turnable knob, a display, such as a liquid crystal (LCD), light-emitting diode (LED), or cathode ray tube (CRT). In some examples the display may be a touch screen. As discussed in this disclosure, processing circuitry 90 may present and receive information relating to electrical stimulation and resulting therapeutic effects via user interface 94. For example, processing circuitry 90 may receive patient input via user interface 94. The input may be, for example, in the form of pressing a button on a keypad or selecting an icon from a touch screen.

Processing circuitry 90 may also present information to the patient in the form of alerts related to delivery of the electrical stimulation to patient 14 or a caregiver via user interface 94. Although not shown, computing device 224 may additionally or alternatively include a data or network interface to another computing device, to facilitate communication with the other device, and presentation of information relating to the electrical stimulation and therapeutic effects after termination of the electrical stimulation via the other device.

Telemetry circuitry 96 supports wireless or wired communication between transesophageal neurostimulation system 210 and computing device 224 under the control of processing circuitry 90. Telemetry circuitry 96 may also be configured to communicate with another computing device via wireless communication techniques, or direct communication through a wired connection. In some examples, telemetry circuitry 96 may be substantially similar to telemetry circuitry 58 of transesophageal neurostimulation system 210 described above, providing wireless communication via an RF or proximal inductive medium. In some examples, telemetry circuitry 96 may include an antenna, which may take on a variety of forms, such as an internal or external antenna.

Examples of local wireless communication techniques that may be employed to facilitate communication between computing device 224 and another computing device include RF communication according to the 802.11 or Bluetooth specification sets, infrared communication, e.g., according to the IrDA standard, or other standard or proprietary telemetry protocols. In this manner, other external devices may be capable of communicating with computing device 224 without needing to establish a secure wireless connection.

Power source 98 delivers operating power to the components of computing device 224. Power source 98 may include a battery and a power generation circuit to produce the operating power. In some examples, the battery may be rechargeable to allow extended operation.

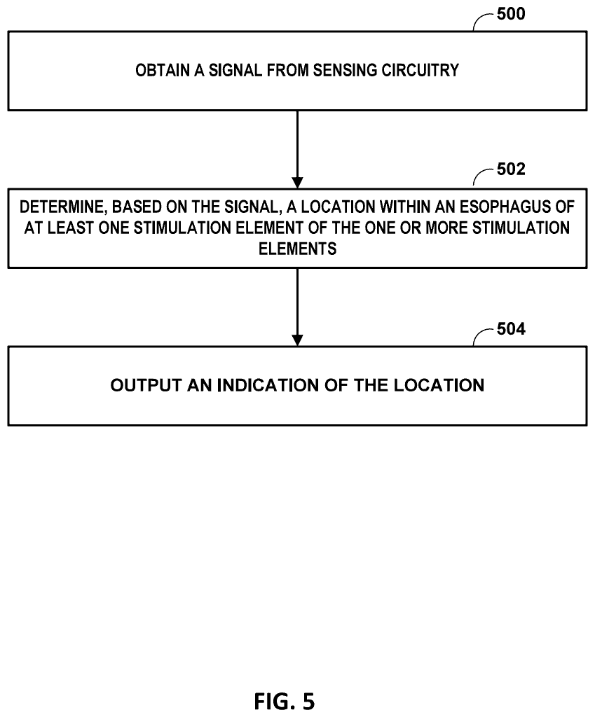

FIG. 5 is a flow diagram illustrating example positioning techniques according to the present disclosure. While discussed primarily with respect to transesophageal neurostimulation system 210 of FIG. 3A, the techniques of FIG. 5 may be performed by other transesophageal neurostimulation systems. Processing circuitry 53 may obtain a signal from sensing circuitry 54 (500). For example, processing circuitry 53 may obtain a signal of the patient from any of electrodes 19 for example, via sensing circuitry 54, and/or any of electrodes 29 (in the case where at least one of the sense electrode is the same as a stimulation element).

Processing circuitry 53 may determine, based on the signal, a location within an esophagus of at least one stimulation element of one or more stimulation elements (502). For example, processing circuitry 53 may utilize the obtained signal to determine a location within esophagus 24 (FIGS. 1 and 2) of one or more stimulation elements, such as stimulation electrodes 29. In the case where the one or more sense electrodes are the one or more stimulation elements, the location of the stimulation element may be directly related to the sensed signal. In the case where the one or more sense electrodes are not the one or more stimulation elements, the location of the one or more stimulation elements in relation to the location of the one or more sense electrodes is known or knowable, and processing circuitry 53 may use a distance between the location of the one or more stimulation elements and the location of the one or more sense electrodes as well as the sensed signal to determine the location of the at least one stimulation element.

Processing circuitry 53 may output an indication based on the location (504). For example, processing circuitry 53 may output an indication of the location, an indication to move the transesophageal neurostimulation device (e.g., so as to move the at least one stimulation element) based on the location, or the like.

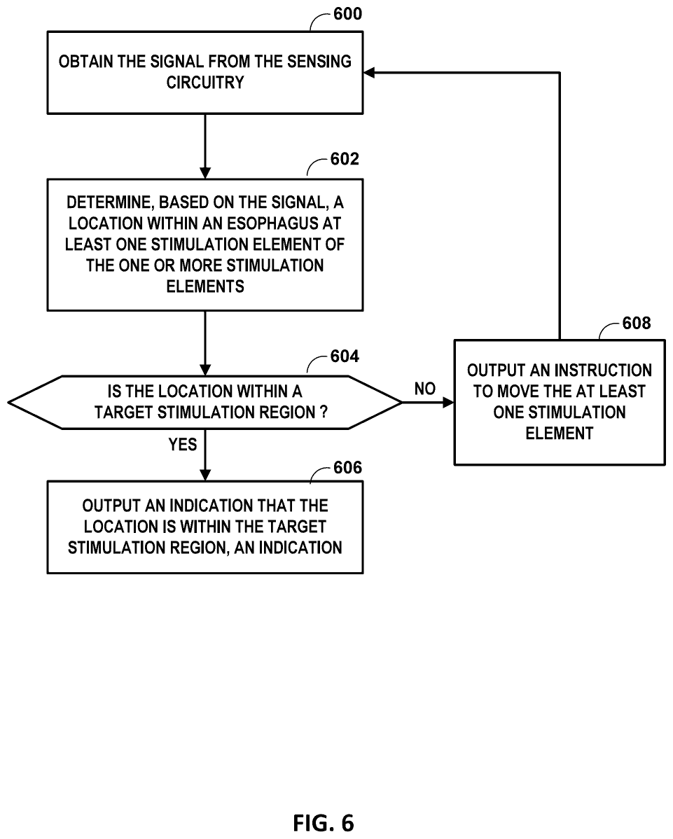

FIG. 6 is a flow diagram illustrating another example of positioning techniques according to one or more aspects of the present disclosure. While discussed primarily with respect to transesophageal neurostimulation system 210 of FIG. 3A, the techniques of FIG. 6 may be performed by other transesophageal neurostimulation systems.

Processing circuitry 53 may obtain a signal from sensing circuitry 54 (600). For example, processing circuitry 53 may obtain a signal of the patient from any of electrodes 19 for example, via sensing circuitry 54, and/or any of electrodes 29 (in the case where at least one of the sense electrode is the same as a stimulation element).

Processing circuitry 53 may determine, based on the signal, a location within an esophagus of at least one stimulation element of one or more stimulation elements (602). For example, processing circuitry 53 may utilize the obtained signal to determine a location within esophagus 24 (FIGS. 1 and 2) of one or more stimulation elements, such as stimulation electrodes 29. In the case where the one or more sense electrodes are the one or more stimulation elements, the location of the stimulation element may be directly related to the sensed signal. In the case where the one or more sense electrodes are not the one or more stimulation elements, the location of the one or more stimulation elements in relation to the location of the one or more sense electrodes is known or knowable, and processing circuitry 53 may use a distance between the location of the one or more stimulation elements and the location of the one or more sense electrodes as well as the sensed signal to determine the location of the at least one stimulation element.

Processing circuitry 53 may determine whether the location is within a target stimulation region (604). For example, processing circuitry 53 may determine whether the location of the one or more stimulation elements is within a region where esophagus 24 passes through diaphragm 18 (FIG. 1).

If the location is within the target stimulation region (the “YES” path from box 604), processing circuitry 53 may output an indication that the location is within the target stimulation region (606). For example, processing circuitry 53 may output a message, for display, that the location is within the target stimulation region, a command to control a start of delivery of the transesophageal stimulation, and/or the like.

If the location is not within the target stimulation region (the “NO” path from box 604), processing circuitry 53 may output an instruction to move the at least one stimulation element (608). For example, processing circuitry 53 may output, for display, an instruction to move the transesophageal neurostimulation device (e.g., so as to move the at least one stimulation element). In this manner, a clinician may be informed to move, e.g., elongated member 30, to move electrodes 34 (both of FIG. 2) so as to better position the at least one stimulation element for transesophageal stimulation. Processing circuitry 53 may then iterate the techniques of FIG. 6 until, for example, the location is within the target stimulation region.

In some examples, the one or more stimulation elements include at least one of a stimulation electrode, a fluid pump, a thermal stimulation element, or a mechanical stimulation element. In some examples, the one or more stimulation elements include at least one stimulation electrode, wherein the stimulation comprises a transesophageal stimulation signal. In some examples, the system further includes one or more memories configured to store stimulation parameters that at least partially define the transesophageal stimulation signal, the one or more memories being communicatively coupled to the processing circuitry. In some examples, the at least one sense electrode comprises the at least one stimulation electrode. For example, a sense electrode may also be a stimulation electrode.

In some examples, as part of determining the location, processing circuitry 53 may determine a morphology of the signal and at least one of compare at least one value associated with the morphology of the signal to at least one threshold or compare the morphology of the signal to a template. In some examples, the signal includes at least one of an electrocardiogram (ECG) signal or an electromyogram (EMG) signal.

In some examples, the indication includes at least one of an instruction to move the at least one stimulation element with respect to a target stimulation region or an indication that the location is acceptable for stimulation. In some examples, the indication that the location is acceptable for stimulation includes a command to control a start of delivery of the transesophageal stimulation. In some examples, the indication that the location is acceptable for stimulation includes a message for display to a user via a user interface. In some examples, the indication includes the instruction to move the at least one stimulation element with respect to the target stimulation region, wherein the target stimulation region comprises a region where the esophagus passes a diaphragm, and wherein the instruction includes an instruction to move the stimulation device a set distance. In some examples, the set distance is in the range of 1 cm to 15 cm, inclusive.

In some examples, processing circuitry 53 may determine whether the location is within a location range, and wherein the indication includes a representation of whether the location is within the location range. In some examples, the location range includes a closest location within the esophagus to a particular anatomy of the patient. In some examples, the particular anatomy includes a left atrium. In some examples, the location range includes a region where the esophagus passes the diaphragm.

In some examples, processing circuitry 53 may, in response to user input obtained from a clinician, control stimulation circuitry 52 to start delivery of the transesophageal stimulation, e.g., to the anatomy of the patient.

In some examples, the location for the at least one stimulation element is a first location of a plurality of locations of the at least one stimulation element due to movement of the at least one stimulation element over time. In some examples, processing circuitry 53 may determine the plurality of locations over time based on the signal over time. Processing circuitry 53 may determine a preferred location among the plurality of locations based on the signal over time. Processing circuitry 53 may output an indication of the preferred location.

The techniques of this disclosure may facilitate the stimulating the cervical, thoracic, or abdominal vagus branches in a manner that is relatively easy and quick to use, such as through transesophageal stimulation. Such techniques may be used for short-term stimulation, such as during an acute health problem, such as surgery or during an abrupt illnesses, such as sepsis, without having to undertake an invasive surgical procedure to implant a vagus nerve stimulation device.

It should be noted that the techniques described herein, may not be limited to treatment or monitoring of a human patient. In alternative examples, the techniques of this disclosure may be applied to non-human patients, e.g., primates, canines, equines, pigs, and felines. These other animals may undergo clinical or research therapies that my benefit from the subject matter of this disclosure.

Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination of the additional features, such as by repeating every feature each time different examples and uses of the stimulation devices are discussed.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processing circuitry” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processing circuitry, programmable processing circuitry, or combinations thereof.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

This disclosure includes the following non-limiting examples.

Example 1. A system comprising: one or more electrodes disposed on a transesophageal stimulation device, the one or more electrodes comprising at least one sense electrode; sensing circuitry configured to generate a signal of a patient via the at least one sense electrode; one or more stimulation elements configured to deliver transesophageal stimulation to anatomy of the patient; and processing circuitry communicatively coupled to the sensing circuitry, the processing circuitry being configured to: obtain the signal from the sensing circuitry; determine, based on the signal, a location within an esophagus of at least one stimulation element of the one or more stimulation elements; and output an indication of the location.

Example 2. The system of example 1, wherein the one or more stimulation elements comprise at least one of a stimulation electrode, a fluid pump, a thermal stimulation element, or a mechanical stimulation element.

Example 3. The system of example 1, wherein the one or more stimulation elements comprise at least one stimulation electrode, wherein the stimulation comprises a transesophageal stimulation signal, the system further comprising one or more memories configured to store stimulation parameters that at least partially define the transesophageal stimulation signal, the one or more memories being communicatively coupled to the processing circuitry.

Example 4. The system of example 3, wherein the at least one sense electrode comprises the at least one stimulation electrode.

Example 5. The system of any of examples 1-4, wherein as part of determining the location, the processing circuitry is configured to: determine a morphology of the signal; and at least one of compare at least one value associated with the morphology of the signal to at least one threshold or compare the morphology of the signal to a template.

Example 6. The system of any of examples 1-5, wherein the signal comprises at least one of an electrocardiogram (ECG) signal or an electromyogram (EMG) signal.

Example 7. The system of any of examples 1-6, wherein the indication comprises at least one of an instruction to move the at least one stimulation element with respect to a target stimulation region or an indication that the location is acceptable for stimulation.

Example 8. The system of example 7, wherein the indication that the location is acceptable for stimulation comprises a command to control a start of delivery of the transesophageal stimulation.

Example 9. The system of example 7 or example 8, wherein the indication that the location is acceptable for stimulation comprises a message for display to a user via a user interface.

Example 10. The system of example 7, wherein the indication comprises the instruction to move the at least one stimulation element with respect to the target stimulation region, wherein the target stimulation region comprises a region where the esophagus passes a diaphragm, and wherein the instruction comprises an instruction to move the stimulation device a set distance.

Example 11. The system of example 10, wherein the set distance is in a range of 1 cm to 15 cm, inclusive.

Example 12. The system of any of examples 1-11, wherein the processing circuitry is further configured to determine whether the location is within a location range, and wherein the indication comprises a representation of whether the location is within the location range.

Example 13. The system of example 12, wherein the location range comprises a closest location within the esophagus to a particular anatomy of the patient.

Example 14. The system of example 13, wherein the particular anatomy comprises a left atrium.

Example 15. The system of example 12, wherein the location range comprises a region where the esophagus passes a diaphragm.

Example 16. The system of any of examples 1-15, wherein the processing circuitry is further configured to, in response to user input obtained from a clinician, control a start of delivery of the transesophageal stimulation.

Example 17. The system of any of examples 1-16, wherein the location for the at least one stimulation element is a first location of a plurality of locations of the at least one stimulation element due to movement of the at least one stimulation element over time, and wherein the processing circuitry is further configured to: determine, based on the signal over time, the plurality of locations; determine, based on the signal over time, a preferred location among the plurality of locations; and output an indication of the preferred location.

Example 18. A method comprising: obtaining, by processing circuitry and from sensing circuitry via at least one sense electrode of one or more electrodes disposed on a transesophageal stimulation device, a signal of a patient; determining, by the processing circuitry and based on the signal, a location of at least one stimulation element of one or more stimulation elements disposed on the transesophageal stimulation device, the at least one stimulation element being configured to deliver transesophageal stimulation to anatomy of the patient; and outputting, by the processing circuitry, an indication of the location.

Example 19. The method of example 18, wherein the signal comprises at least one of an electrocardiogram (ECG) signal or an electromyogram (EMG) signal.

Example 20. A transesophageal neurostimulation system comprising: one or more electrodes disposed on a transesophageal stimulation device, the one or more electrodes comprising at least one sense electrode and at least one stimulation electrode configured to deliver a transesophageal stimulation signal to anatomy of a patient; sensing circuitry configured to generate a patient signal of the patient via the at least one sense electrode; stimulation circuitry configured to generate the transesophageal stimulation signal; one or more memories configured to store stimulation parameters that at least partially define the transesophageal stimulation signal; and processing circuitry communicatively coupled to the one or more memories, the sensing circuitry, and the stimulation circuitry, the processing circuitry being configured to: obtain the patient signal from the at least one sense electrode; determine, based on the patient signal, a location within an esophagus for delivering the transesophageal stimulation signal from the at least one stimulation electrode; and output an indication based on the location.

Various examples have been described herein. Any combination of the described operations or functions is contemplated. These and other examples are within the scope of the following claims. Based upon the above discussion and illustrations, it is recognized that various modifications and changes may be made to the disclosed examples in a manner that does not require strictly adherence to the examples and applications illustrated and described herein. Such modifications do not depart from the true spirit and scope of various aspects of the disclosure, including aspects set forth in the claims.

What is claimed is:

1. A system comprising:
one or more electrodes disposed on a transesophageal stimulation device, the one or more electrodes comprising at least one sense electrode;
sensing circuitry configured to generate a signal of a patient via the at least one sense electrode;
one or more stimulation elements configured to deliver transesophageal stimulation to anatomy of the patient; and
processing circuitry communicatively coupled to the sensing circuitry, the processing circuitry being configured to:
obtain the signal from the sensing circuitry;
determine, based on the signal, a location within an esophagus of at least one stimulation element of the one or more stimulation elements; and
output an indication of the location.

2. The system of claim 1, wherein the one or more stimulation elements comprise at least one of a stimulation electrode, a fluid pump, a thermal stimulation element, or a mechanical stimulation element.

3. The system of claim 1, wherein the one or more stimulation elements comprise at least one stimulation electrode, wherein the stimulation comprises a transesophageal stimulation signal, the system further comprising one or more memories configured to store stimulation parameters that at least partially define the transesophageal stimulation signal, the one or more memories being communicatively coupled to the processing circuitry.

4. The system of claim 3, wherein the at least one sense electrode comprises the at least one stimulation electrode.

5. The system of claim 1, wherein as part of determining the location, the processing circuitry is configured to:
determine a morphology of the signal; and
at least one of compare at least one value associated with the morphology of the signal to at least one threshold or compare the morphology of the signal to a template.

6. The system of claim 1, wherein the signal comprises at least one of an electrocardiogram (ECG) signal or an electromyogram (EMG) signal.

7. The system of claim 1, wherein the indication comprises at least one of an instruction to move the at least one stimulation element with respect to a target stimulation region or an indication that the location is acceptable for stimulation.

8. The system of claim 7, wherein the indication that the location is acceptable for stimulation comprises a command to control a start of delivery of the transesophageal stimulation.

9. The system of claim 7, wherein the indication that the location is acceptable for stimulation comprises a message for display to a user via a user interface.

10. The system of claim 7, wherein the indication comprises the instruction to move the at least one stimulation element with respect to the target stimulation region, wherein the target stimulation region comprises a region where the esophagus passes a diaphragm, and wherein the instruction comprises an instruction to move the stimulation device a set distance.

11. The system of claim 10, wherein the set distance is in a range of 1 cm to 15 cm, inclusive.

12. The system of claim 1, wherein the processing circuitry is further configured to determine whether the location is within a location range, and wherein the indication comprises a representation of whether the location is within the location range.

13. The system of claim 12, wherein the location range comprises a closest location within the esophagus to a particular anatomy of the patient.

14. The system of claim 13, wherein the particular anatomy comprises a left atrium.

15. The system of claim 12, wherein the location range comprises a region where the esophagus passes a diaphragm.

16. The system of claim 15, wherein the processing circuitry is further configured to, in response to user input obtained from a clinician, control a start of delivery of the transesophageal stimulation.

17. The system of claim 16, wherein the location for the at least one stimulation element is a first location of a plurality of locations of the at least one stimulation element due to movement of the at least one stimulation element over time, and wherein the processing circuitry is further configured to:
determine, based on the signal over time, the plurality of locations;
determine, based on the signal over time, a preferred location among the plurality of locations; and
output an indication of the preferred location.

18. A method comprising:
obtaining, by processing circuitry and from sensing circuitry via at least one sense electrode of one or more electrodes disposed on a transesophageal stimulation device, a signal of a patient;
determining, by the processing circuitry and based on the signal, a location of at least one stimulation element of one or more stimulation elements disposed on the transesophageal stimulation device, the at least one stimulation element being configured to deliver transesophageal stimulation to anatomy of the patient; and
outputting, by the processing circuitry, an indication of the location.

19. The method of claim 18, wherein the signal comprises at least one of an electrocardiogram (ECG) signal or an electromyogram (EMG) signal.

20. A transesophageal neurostimulation device comprising:
one or more electrodes disposed on a transesophageal stimulation device, the one or more electrodes comprising at least one sense electrode and at least one stimulation electrode configured to deliver a transesophageal stimulation signal to anatomy of a patient;
sensing circuitry configured to generate a patient signal of the patient via the at least one sense electrode;
stimulation circuitry configured to generate the transesophageal stimulation signal;
one or more memories configured to store stimulation parameters that at least partially define the transesophageal stimulation signal; and
processing circuitry communicatively coupled to the one or more memories, the sensing circuitry, and the stimulation circuitry, the processing circuitry being configured to:
obtain the patient signal from the at least one sense electrode;
determine, based on the patient signal, a location within an esophagus for delivering the transesophageal stimulation signal from the at least one stimulation electrode; and
output an indication based on the location.
